(19) United States
(12) Patent Application Publication
(10) Pub. No.: US 2018/0051056 A1
(43) Pub. Date: Feb. 22, 2018

(54) SYNTHETIC MINIATURE PROTEIN SCAFFOLDS, PHARMACEUTICAL COMPOSITIONS AND METHODS OF USING SAME

(71) Applicants: Kent Kirshenbaum, New York, NY (US); Timothy Craven, Randolph, NJ (US)

(72) Inventors: Kent Kirshenbaum, New York, NY (US); Timothy Craven, Randolph, NJ (US)

(21) Appl. No.: 15/678,204

(22) Filed: Aug. 16, 2017

Related U.S. Application Data
(60) Provisional application No. 62/375,678, filed on Aug. 16, 2016.

(51) Int. Cl. C07K 7/08

(52) U.S. Cl. CPC C07K 7/08

(57) ABSTRACT

Synthetic miniature protein scaffolds are described herein, as are compositions comprising same. Methods for the preparation of the synthetic miniature protein scaffolds and compositions thereof, and their use in preventing and/or treating conditions relating to diseases or disorders related to or associated with protein-protein interactions directly mediated by poly-proline type-II helix and/or beta-strand interactions are also encompassed herein.

### GOVERNMENTAL SUPPORT

This invention was made with government support under grant CHE-1507946 awarded by the National Science Foundation. The government has certain rights in the invention.

### FIELD OF THE INVENTION

Poly-proline type-II (PPII) helical “PXXP motifs” are the recognition elements for a variety of protein-protein interactions critical for cellular signaling. Pursuant to the objective of designing and generating synthetic miniature protein scaffolds that serve as PPII-helical peptidomimetics and PPII-helical/beta-strand peptidomimetics, novel synthetic miniature protein scaffolds are described herein, as are compositions comprising same. The invention also relates to methods for the preparation of the synthetic miniature protein scaffolds and compositions thereof, and their use in preventing and/or treating conditions relating to diseases or disorders related to or associated with protein-protein interactions directly mediated by poly-proline type-II helix and/or beta-strand interactions.

### BACKGROUND OF THE INVENTION

In the field of peptidomimetics research, extensive efforts have been made to recapitulate the structural features present in naturally occurring bioactive peptides (Ripka et al. Curr. Opin. in Chem. Bio. 1998, 2, 441-452; Steer et al. Curr. Med. Chem. 2002, 9, 811-822; Patch et al. Curr. Opin. In Chem. Biol. 2002, 6, 872-877). Many functional peptidomimetics such as magainin mimics (Liu et al. J. Am. Chem. Soc. 2001, 123, 7553-7559; Wieprecht et al. Biochemistry 1996, 35, 10844-10853; Porter et al. J. Am. Chem. Soc. 2005, 127, 11516-11529; Numao et al. Biol. Pharm. Bull. 1997, 20, 800-804; Rennie et al. J. Ind. Microbiol. Biotechnol. 2005, 32, 296-300), integrin mimics (Pasqualini et al. J. Cell Biol. 1995, 130, 1189-1196; Scarborough et al. Curr. Med. Chem. 1999, 6, 971-981) and somatostatin mimics (Gademann et al. J. Med. Chem. 2001, 44, 2460-2468; Gademann et al. Helv. Chim. Acta 2000, 83, 16-33) highlight the significance of structural mimicry for their function. More recently, efforts have been made to enhance the conformational ordering of peptidomimetic oligomers (Fink et al. J. Am. Chem. Soc. 1998, 120, 4334-4344; Phillips et al. J. Am. Chem. Soc. 2002, 124, 58-66; Abell et al. Lett. Pept. Sci. 2001, 8, 267-272; Clark et al. J. Am. Chem. Soc. 1995, 117, 12364-12365; Dimartino et al. Org. Lett. 2005, 7, 2389-2392). Stabilizing or rigidifying polymer conformations may lead to enhanced binding affinities (Sewald et al., Peptides: Chemistry and Biology. Wiley-VCH: Weinheim, Germany: 2002; Wipf. Chem. Rev. 1995, 95, 2115-2134). To this end, several methods have been developed to enhance the conformational ordering of non-natural polymers (Sewald et al., Peptides: Chemistry and Biology. Wiley-VCH: Weinheim, Germany: 2002; Wipf. Chem. Rev. 1995, 95, 2115-2134; Holub et al. Org. Lett. 2007, 9, 3275-3278). These methods include the introduction of both covalent and non-covalent intramolecular interactions. Some examples of covalent constraints include site-specific macrocyclization via Huisgen 1,3-dipolar cycloaddition (Holub et al. Org. Lett. 2007, 9, 3275-3278), head-to-tail macrocyclization (Gademann et al. Angew. Chem., Int. 1999, 38, 1223-1226; Robinson et al. Bioorg. Med. Chem. 2005, 13, 2055-2064; Wels et al. Bioorg. Med. Chem. Lett. 2005, 15, 287-290; Shankaramma et al. Chem. Commun. 2003, 1842-1843; Locardi et al. J. Am. Chem. Soc. 2001, 123, 8189-8196; Chakraborty et al. J. Org. Chem. 2003, 68, 6257-6263; Angell et al. J. Org. Chem. 2005, 70, 9595-9598; Norgren et al. J. Org. Chem. 2006, 71, 6814-6821; Clark et al. J. Am. Chem. Soc. 1998, 120, 651-656; Yuan et al. J. Am. Chem. Soc. 2004, 126, 11120-11121; Nnanabu et al. Org. Lett. 2006, 8, 1259-62; Jiang et al. Org. Lett. 2004, 6, 2985-2988; Mann et al. Org. Lett. 2003, 5, 4567-4570; Wels et al. Org. Lett. 2002, 4, 2173-2176; Bru et al. Tetrahedron Lett. 2005, 46, 7781-7785; Vaz et al. Org. Lett. 2006, 8, 4199-4202; Buttner et al. Chem. Eur. J. 2005, 11, 6145-6158; Royo et al. Tetrahedron Lett. 2002, 43, 2029-2032) and generation of hydrogen bond surrogates via metathesis reactions (Dimartino et al. Org. Lett. 2005, 7, 2389-2392).

Peptoids, for example, are a class of peptidomimetics which comprise N-substituted glycine monomer units (Figliozzi et al, Synthesis of N-substituted glycine peptoid libraries. In Methods Enzymol., Academic Press: 1996; Vol. 267, pp 437-447; Bartlett et al., Proc. Natl. Acad. Sci. U.S.A. 1992, 89, 9367-9371; the entire content of which is incorporated herein by reference). Peptoids are an important class of sequence-specific peptidomimetics shown to generate diverse biological activities (Patch et al. In Pseudo-peptides in Drug Development; Nielson, P. E., Ed.; Wiley-VCH: Weinheim, Germany, 2004; pp 1-35; Miller et al. Drug Dev. Res. 1995, 35, 20-32; Murphy et al. Proc. Natl. Acad. Sci. U.S.A. 1998, 95, 1517-1522; Nguyen et al. Science 1998, 282, 2088-2092; Ng et al. Bioorg. Med. Chem. 1999, 7, 1781-1785; Patch et al. J. Am. Chem. Soc. 2003, 125, 12092-12093; Wender et al. Proc. Natl. Acad. Sci. U.S.A. 2000, 97, 13003-13008; Wu et al. Chem. Biol. 2003, 10, 1057-1063; Chongsiriwatana et al. Proc. Natl. Acad. Sci. U.S.S. 2008, 105, 2794-2799). Oligopeptoids can be designed to display chemical moieties analogous to the bioactive peptide side chains while their abiotic backbones provide protection from proteolytic degradation.

Peptoid sequences comprised of bulky branched side chains have the capacity to adopt a stable helical secondary structure, although some conformational heterogeneity is evident in solution (Armand et al. Proc. Natl. Acad. Sci. US.A. 1998, 95, 4309-4314; Kirshenbaum et al. Proc. Natl. Acad. Sci. U.S.A. 1998, 95, 4303-4308; Wu et al. J. Am. Chem. Soc. 2003, 125, 13525-13530). The crystal structure of a linear peptoid homopentamer composed of bulky branched side chains exhibits a helical conformation resembling that of a polyproline type I helix (Armand et al. Proc. Natl. Acad. Sci. U.S.A. 1998, 95, 4309-4314; Kirshenbaum et al. Proc. Natl. Acad. Sci. U.S.A. 1998, 95, 4303-4308; Wu et al. J. Am. Chem. Soc. 2003, 125, 13525-13530). Oligopeptoid sequences incorporating repeating units of two bulky branched side chains and a cationic side chain form facially amphiphilic helical structures. Recent studies describe antimicrobial activities generated from facially amphiphilic helical peptoids (Patch et al. J. Am. Chem. Soc. 2003, 125, 12092-12093; Chongsiriwatana et al. Proc. Natl. Acad. Sci. U.S.S. 2008, 105, 2794-2799). These peptoid oligomers are reported to be good functional mimics of maganin-2 amide, a peptide antimicrobial agent from Xenopus skin (Patch et al. J. Am. Chem. Soc. 2003, 125, 12092-12093; Zasloff. Proc. Natl. Acad. Sci. USA 1987, 84, 5449-5453).

Peptoid sequences having an energetic preference for PPI or PPII secondary structures have been designed, but such designs have been limited to specific side-chain types [Shah et al. (2008 J. Am. Chem. Soc. 130:166222-16632); Kirshenbaum et al. (1998, Proc. Natl. Acad. Sci. U.S.A. 95:4303-4308); Stringer et al. (2011, J. Am. Chem. Soc. 133:15559-15567); the entire content of each of which is incorporated herein by reference]. Accordingly, such sequences are of limited utility, due at least in part to the limited array of side-chain types that may be employed, and are thus not readily adapted to applications wherein customization is desired.

Advances have been made with regard to the generation of protein-like oligomers with heterogeneous backbones as detailed in, for example, Reinert et al. (2014, Chem Sci 5:3325-3330) and Tavenor et al. (2016. Chein Commun 52:3789-3792), the entire content of each of which is incorporated herein by reference. These references relate to substitutions in an alpha-helicle region of a protein of interest. Folded miniature proteins having thermostable tertiary structures have also been described by Craven et al. (2016, J Am Chem Soc 138:1543-1550), the entire content of which is incorporated herein by reference.

### SUMMARY OF THE INVENTION

Many protein-protein interactions critical for cellular signaling are mediated through poly-proline type-II (PPII) helical “PXXP motifs” and/or beta-strands. Despite development of protocols for locking peptides into α-helical and β-strand conformations, there remains a lack of analogous methods for generating mimics of PPII-helical structures. The present inventors herein describe a strategy to enforce PPII-helical secondary structure in the 19 residue “TrpPlexus” miniature protein. Through sequence variation, the present inventors show that a network of cation-n interactions can drive the formation of PPII-helical conformations for both peptide and N-substituted glycine “peptoid” residues/monomers. The achievement of chemically diverse PPII-helical scaffolds provides a new route towards discovering peptidomimetic inhibitors of protein-protein interactions mediated by PXXP motifs.

These findings, therefore, offer novel synthetic protein scaffolds that are promising candidates for therapeutic use. Pharmaceutical compositions comprising these synthetic protein scaffolds as active ingredients and their use to treat, prevent or ameliorate a range of diseases or disorders in mammals of various genesis or etiology are also envisioned. In a particular embodiment, synthetic protein scaffolds described herein and pharmaceutical compositions comprising same are used to treat, prevent, or ameliorate diseases or disorders related to or associated with protein-protein interactions directly mediated by poly-proline type-II helix interactions.

More particularly, the present invention relates to synthetic miniature protein scaffolds (also referred to herein as synthetic PPII-helical scaffolds, synthetic protein scaffolds, or protein scaffolds) that serve as peptidomimetics for PPII-helices and beta-strands operably linked thereto.

In accordance with the discoveries of the present inventors, a miniature protein scaffold comprising Arg Val Arg Val Arg Thr Ser Arg Xaa Gly Tyr Asn Gly Thr Trp Ser Glu Trp Ser is envisioned, wherein Xaa at position 9 is proline (Pro) or D-Pro and at least one of the arginine (Arg) at position 1, 4, or 8; the valine (Val) at position 2 or 4; the threonine (Thr) at position 6 or 14; the serine (Ser) at position 7, 16, or 19; the tyrosine (Tyr) at position 11; the asparagine (Asn) at position 12; the glycine (Gly) at position 13; and the glutamic acid (Glu) at position 17; is replaced by an abiotic monomer, provided that the abiotic monomer is other than D-Pro or a substituted Pro residue.

Also encompassed herein is a miniature protein scaffold comprising Arg Cys Arg Val Arg Thr Ser Arg D-Pro Gly Tyr Asn Gly Thr Trp Ser Glu Trp Cys, wherein at least one of the arginine (Arg) at position 1, 4, or 8; the valine (Val) at position 4; the threonine (Thr) at position 6 or 14; the serine (Ser) at position 7 or 16; the tyrosine (Tyr) at position 11; the asparagine (Asn) at position 12; the glycine (Gly) at position 13; and the glutamic acid (Glu) at position 17; is replaced by an abiotic monomer, provided that the abiotic monomer is other than D-Pro or a substituted Pro residue. In a particular embodiment thereof, the cysteines (Cys) at positions 2 and 19 are linked via a disulfide bridge.

In one aspect, the miniature protein scaffold comprises at least one abiotic monomer that is selected from an N-substituted glycine (also known as an N-substituted glycine peptoid or peptoid), an N-substituted amino acid (also described as a peptide tertiary amide), a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

In a particular embodiment, the miniature protein scaffold comprises at least one abiotic monomer selected from an N-substituted glycine, a beta-peptoid, a beta-peptide, and a gamma-peptide. In a more particular embodiment, the at least one abiotic monomer is an N-substituted glycine.

In a more particular embodiment, at least one of the threonine (Thr) at position 14 and the glutamic acid(Glu) at position 17 is replaced by an abiotic monomer in the miniature protein scaffold.

In a still more particular embodiment, the Thr at position 14 is replaced by an N-substituted glycine or an N-substituted amino acid in the miniature protein scaffold.

In another particular embodiment, the Glu at position 17 is replaced by an N-substituted glycine or an N-substituted amino acid in the miniature protein scaffold.

In yet another particular embodiment, the Thr at position 14 and the Glu at position 17 are replaced by an N-substituted glycine or an N-substituted amino acid in the miniature protein scaffold.

In an aspect thereof, the at least one abiotic monomer included in the miniature protein scaffold is an N-substituted glycine such as any one of those described in U.S. Pat. No. 8,828,413; U.S. Application Publication Nos. 2014/0274916 and 2015/0044189; and Culf et al. (2010, Molecules 15:5282-5335), the entire content of each of which is incorporated herein by reference.

In a particular embodiment thereof, the abiotic monomer is an N-substituted glycine selected from the group consisting of N-(methoxyethyl) glycine, N—(S/R)-phenylethylamine glycine, N-(aryl) glycine, and N-(alkyl) glycine. In a more particular embodiment, the abiotic monomer is N-methoxyethyl glycine.

In another particular embodiment, the miniature protein scaffold comprises Arg Cys Arg Val Arg Thr Ser Arg D-Pro Gly Tyr Asn Gly Xaa Trp Ser Glu Trp Cys, wherein Xaa is N-methoxyethyl glycine.

In another particular embodiment, the miniature protein scaffold comprises Arg Cys Arg Val Arg Thr Ser Arg D-Pro Gly Tyr Asn Gly Thr Trp Ser Xaa Trp Cys, wherein Xaa is N-methoxyethyl glycine.

In another particular embodiment, the miniature protein scaffold comprises Arg Cys Arg Val Arg Thr Ser Arg D-Pro Gly Tyr Asn Gly Pro Trp Ser Xaa Trp Cys, wherein Xaa is N-methoxyethyl glycine.

In another particular embodiment, the miniature protein scaffold comprises Arg Cys Arg Val Arg Thr Ser Arg D-Pro Gly Tyr Asn Gly Xaa Trp Ser Pro Trp Cys, wherein Xaa is N-methoxyethyl glycine.

In another particular embodiment, the miniature protein scaffold comprises Arg Cys Arg Val Arg Thr Ser Arg D-Pro Gly Tyr Asn Gly Xaa Trp Ser Xaa Trp Cys, wherein Xaa is N-methoxyethyl glycine.

In another particular embodiment, the miniature protein scaffold comprises Arg Val Arg Val Arg Thr Ser Arg D-Pro Gly Tyr Asn Gly Xaa Trp Ser Glu Trp Ser, wherein Xaa is N-methoxyethyl glycine.

In another particular embodiment, the miniature protein scaffold comprises Arg Val Arg Val Arg Thr Ser Arg D-Pro Gly Tyr Asn Gly Thr Trp Ser Xaa Trp Ser, wherein Xaa is N-methoxyethyl glycine.

In another particular embodiment, the miniature protein scaffold comprises Arg Val Arg Val Arg Thr Ser Arg D-Pro Gly Tyr Asn Gly Pro Trp Ser Xaa Trp Ser, wherein Xaa is N-methoxyethyl glycine.

In another particular embodiment, the miniature protein scaffold comprises Arg Val Arg Val Arg Thr Ser Arg D-Pro Gly Tyr Asn Gly Xaa Trp Ser Pro Trp Ser, wherein Xaa is N-methoxyethyl glycine.

In another particular embodiment, the miniature protein scaffold comprises Arg Val Arg Val Arg Thr Ser Arg D-Pro Gly Tyr Asn Gly Xaa Trp Ser Xaa Trp Ser, wherein Xaa is N-methoxyethyl glycine.

In an aspect thereof, the at least one abiotic monomer included in the miniature protein scaffold is an N-substituted amino acid selected from the group consisting of N-methyl alanine, N-methyl phenylalanine, N-methyl valine, N-methyl leucine, N-methyl isoleucine, N-methyl tryptophan, N-methyl methionine, N-methyl aspartic acid, N-methyl glutamic acid, N-methyl glycine, N-methyl serine, N-methyl threonine, N-methyl cysteine, N-methyl tyrosine, N-methyl asparagine, N-methyl glutamine, N-methyl lysine, N-methyl arginine, or N-methyl histidine.

N-substituted amino acids are known in the art, as are methods for making same. See, for example, Pels et al. (2015, ACS Comb Sci 17:152-155), the entire content of which is incorporated herein by reference.

In an aspect thereof, the at least one abiotic monomer included in the miniature protein scaffold is a side chain variant of Pro such as any one of those described in Pandey et al. (2013, J Am Chem Soc 135:4333-4363); Bach et al. (2013, Applied Microbiology and Biotechnology 97:6623-6634); Sigma Aldrich technical documents (available prior to Jun. 30, 2016) Chemfiles 5: Article 12, “Proline Derivatives and Analogs”; Anaspec Product catalog (available prior to Jun. 30, 2016), “Analogs of Proline”, the entire content of each of which is incorporated herein by reference.

In a particular embodiment thereof, the side chain variant is 4-fluoro-proline, 3-hydroxy-proline, 4-cyano-proline, 4-amino-proline, 3,4-dehydro-proline, azetidine-2-carboxylic acid, pipecolic acid, 4-oxa-proline, 3-thia-proline, or 4-[2-(trifluoromethyl)benzyl]-proline.

In a further aspect, a miniature protein scaffold comprising Arg Cys Arg Val Arg Thr Ser Arg Xaa Gly Tyr Asn Gly Thr Trp Ser Glu Trp Cys is envisioned, wherein Xaa is proline (Pro) or D-Pro, the cysteines (Cys) at positions 2 and 19 are optionally linked via a disulfide bridge, and at least one of the threonine (Thr) at position 14 and the glutamic acid( Glu) at position 17 is replaced by an abiotic monomer, provided that the abiotic monomer is other than D-Pro or a substituted Pro residue.

In a particular embodiment thereof with respect to the miniature protein scaffold comprising Arg Cys Arg Val Arg Thr Ser Arg Xaa Gly Tyr Asn Gly Thr Trp Ser Glu Trp Cys, the Thr at position 14 is replaced by an abiotic monomer selected from the group consisting of an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

In another particular embodiment thereof with respect to the miniature protein scaffold comprising Arg Cys Arg Val Arg Thr Ser Arg Xaa Gly Tyr Asn Gly Thr Trp Ser Glu Trp Cys, the Glu at position 17 is replaced by an abiotic monomer selected from the group consisting of an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

In a still more particular embodiment thereof with respect to the miniature protein scaffold comprising Arg Cys Arg Val Arg Thr Ser Arg Xaa Gly Tyr Asn Gly Thr Trp Ser Glu Trp Cys, the Thr at position 14 and the Glu at position 17 are replaced by an abiotic monomer selected from the group consisting of an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

In another particular embodiment thereof with respect to the miniature protein scaffold comprising Arg Cys Arg Val Arg Thr Ser Arg Xaa Gly Tyr Asn Gly Thr Trp Ser Glu Trp Cys, the abiotic monomer is selected from an N-substituted glycine, a beta-peptoid, a beta-peptide, and a gamma-peptide. In a still more particular embodiment, the abiotic monomer is an N-substituted glycine. Exemplary N-substituted glycine peptoids include N-(methoxyethyl) glycine, N-(S/R)-phenylethylamine glycine, N-(aryl) glycine, and N-(alkyl) glycine.

In another aspect with respect to the miniature protein scaffold comprising Arg Cys Arg Val Arg Thr Ser Arg Xaa Gly Tyr Asn Gly Thr Trp Ser Glu Trp Cys, the abiotic monomer is an N-substituted amino acid, including any one of N-methyl alanine, N-methyl phenylalanine, N-methyl valine, N-methyl leucine, N-methyl isoleucine, N-methyl tryptophan, N-methyl methionine, N-methyl aspartic acid, N-methyl glutamic acid, N-methyl serine, N-methyl threonine, N-methyl cysteine, N-methyl tyrosine, N-methyl asparagine, N-methyl glutamine, N-methyl lysine, N-methyl arginine, and N-methyl histidine. As described herein, N-substituted amino acids are known in the art, as are methods for making same. See, for example, Pels et al. (2015, ACS Comb Sci 17:152-155), the entire content of which is incorporated herein by reference.

In another aspect with respect to the miniature protein scaffold comprising Arg Cys Arg Val Arg Thr Ser Arg Xaa Gly Tyr Asn Gly Thr Trp Ser Glu Trp Cys, the abiotic monomer is a side chain variant of Pro. Side chain variant of Pro are known in the art and described in, for example, Pandey et al. (2013, J Am Chem Soc 135:4333-4363); Bach et al. (2013, Applied Microbiology and Biotechnology 97:6623-6634); Sigma Aldrich technical documents (available prior to Jun. 30, 2016) Chemfiles 5: Article 12, “Proline Derivatives and Analogs”; Anaspec Product catalog (available prior to Jun. 30, 2016), “Analogs of Proline”, the entire content of each of which is incorporated herein by reference. Exemplary side chain variants of Pro envisioned herein include 4-fluoro-proline, 3-hydroxy-proline, 4-cyano-proline, 4-amino-proline, 3,4-dehydro-proline, azetidine-2-carboxylic acid, pipecolic acid, 4-oxa-proline, 3-thia-proline, or 4-[2-(trifluoromethyl)benzyl]-proline.

In embodiments of the miniature protein scaffold wherein the abiotic monomer is an N-substituted glycine or peptoid monomer, the peptoid monomers have the following structure:

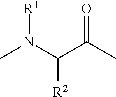

wherein a peptoid monomer may be according to formula IIa, IIb, IIc, or IId:

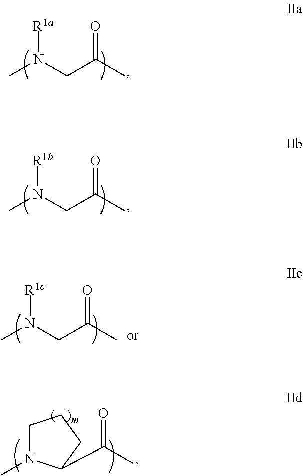

wherein

each R¹ is independently substituted or unsubstituted alkyl;

each R² is independently hydrogen, or substituted or unsubstituted alkyl; or each R¹ and R² are join together to form a 4-7 membered heterocyclic ring;

each R^{1a} is independently unsubstituted alkyl;

each R^{1b} is independently aminoalkyl, guanidinoalkyl (H₂N—C(═NH)—NH-alkyl), or N-containing heteroarylalkyl;

each R^{1c} is independently substituted or unsubstituted aryl, substituted or unsubstituted aralkyl, substituted or unsubstituted heteroarylalkyl,or substituted or unsubstituted diarylalkyl;

each m is 0, 1, 2, or 3;

or a salt thereof; and stereoisomers, isotopic variants and tautomers thereof.

In one embodiment, with respect to the miniature protein scaffold described herein, the abiotic monomer is a peptoid wherein R^{1b} is 4-aminobutyl, 2-imidazol-4-ylethyl, aminoalkyl, aminomethyl, 2-aminoethyl, 3-aminopropyl, or 6-aminohexyl.

In a further aspect, the present invention provides a method for the preparation of the protein scaffolds of the invention.

In a further aspect, miniature protein scaffolds desribed herein may be used to prevent or treat conditions relating to diseases or disorders related to or associated with protein-protein interactions directly mediated by poly-proline type-II helix and/or beta-strand interactions. The miniature protein scaffolds could be designed and assembled to include a abiotic monomer pertinent for the treatment of a particular disease or condition of interest by way of the ability of the protein scaffold to inhibit a protein-protein interaction that is in part mediated by a poly-proline type-II helix and/or beta-strand, and then formulated into appropriate compositions and dosage forms for administration or application to an affected host. Moreover, such compositions may comprise the miniature protein scaffolds of the invention in mixtures or combinations with other therapeutic agents of utility for treating the disease or condition. In such formulations, the miniature protein scaffolds of the invention may act synergistically with the other therapeutic agents, so that the resulting composition demonstrates improved effectiveness.

The invention also relates to methods for the preparation of the synthetic miniature protein scaffolds and compositions thereof, and their use in preventing and/or treating conditions relating to diseases or disorders related to or associated with protein-protein interactions directly mediated by poly-proline type-II helix nad/or beta-strand interactions.

Other objects and advantages will become apparent to those skilled in the art from a consideration of the ensuing detailed description, which proceeds with reference to the following illustrative drawings.

### BRIEF DESCRIPTION OF THE DRAWINGS

FIG. 1A-D. The cyclic TrpPlexus miniature protein scaffold as a mimetic of PXXP and beta-strand motifs and beta-strand. (1A) Sequence and secondary structure of the cyclic TrpPlexus scaffold. A lower-case “p” denotes a D-proline residue. (1B) Cartoon representation of the scaffold highlighting the co-facial i and i+3 PPII-helical residues in red. The central cation-π interaction network is highlighted by +'s and π's connected by dotted lines. The disulfide-bridge is highlighted in yellow. (1C) A representative PXXP motif binding protein, the SH3 domain of Crk (PDBid: 1CKA) is depicted in grey with its PXXP motif ligand in red (proline) and purple. (1D) Model of the cyclic TrpPlexus scaffold mimicking the proline placements of PXXP motifs.

FIG. 2A-C, Sequences, CD spectra, and thermal melts for the nine disulfide-bridged miniature protein sequences. The cTp_TE sequence refers to the “native” TrpPlexus. Units are in mean residue ellipticity (MRF, deg·m²·dmol⁻¹·res⁻¹). The colors of the solid and dashed lines in the top panel identify each sequence in the subsequent plots. (2A) Far-UV CD spectra of cyclic TrpPlexus sequences (100 μM, 1 mm, path length, 10 mM PBS buffer, pH 7.5, 25° C.). (2B) Far-UV CD thermal melts monitoring mean residue ellipticity at 228 nm as a function of temperature for sequences exhibiting PPII-helical content (100 μM, 1 mm path length, 10 mM PBS buffer, pH 7.5), (2C) Near-UV CD spectra of cyclic TrpPlexus sequences (50 μM, 1 cm path length, 10 mM PBS buffer, pH 7.5, 25° C.). Note: Spectra for sequences represented by solid lines were confirmed to achieve the TrpPlexus fold dashed lines represent sequences that did not.

FIG. 3. Representative analytical HPLC traces (5→95% ACN/H₂O gradient in 10 minutes, C18 column) for sequences cTp_TE, cTp₁₃ XE, and cTp_TX tracking conversion of the linear to the cyclic disulfide-bridged peptides. The cyclization conditions were 20:1 H₂O:DMSO, pH 3.8, 4 ° C., and ˜2 mM concentration. Black and orange asterisks mark the peaks corresponding to the reduced linear and oxidized cyclic masses, respectively. Note: An “X” in a sequences denotes an “Ninaeo” residue.

FIG. 4. ¹H—¹H NOESY spectra of select sequences highlighting the critical NOEs (dashed lines in left panel) that support the cation-H interaction network (green and purple boxes, top panel), PPII-helix:β-strand interface, and disulfide-bridge structure (bottom panel). All 2D spectra are presented in units of ppm (6) in the F1 and F2 dimensions. ¹H—¹H NOESY spectra were collected using a 275 ms mixing time, pH 3.8, ˜1.8 mM, and 4° C. The boxed spectra correspond to the sequences identified at the top of each column. A green checked box or a red “X” indicates whether or not the sequence was determined to be folded. Note: Dashed vertical lines in ¹H—¹H NOESY spectra identify side-chains having multiple sets of chemical shifts.

FIG. 5. Model structures in agreement with both observed long-range unambiguous NOEs as well as near and far-UV CD spectra. The central PPII-helix is highlighted in purple, the residue types at positions i and i+3 in red, and the disulfide-bridge in yellow. Tryptophan rotamers are modeled as m95° unless otherwise noted as m-90°.

### DETAILED DESCRIPTION OF THE INVENTION

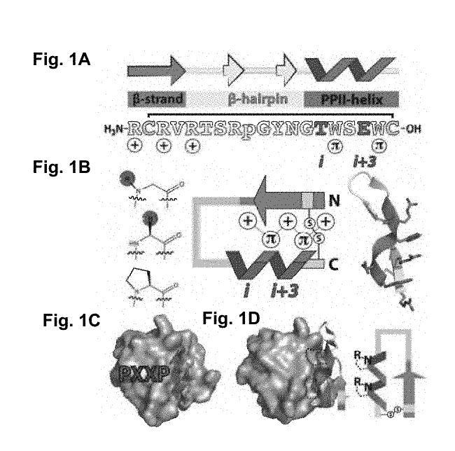

The left-handed poly-proline type-II (PPII) helix is a distinctive secondary structure type that is not defined by a propagated network of hydrogen bonds. Consequently, it is difficult to stabilize PPII-helices by side-chain “stapling” or other types of covalent constraints. Proline-rich PPII-helices are ligands for SH3, WW, EVH1, UEV, GYF, and Profilin domains. Several proteins containing these domains have been found to bind “PXXP motifs” (where P is always a proline residue and X can be other amino acid types), which mediate their protein-protein interactions. PPII-helices feature three residues per turn, orienting the residues at positions i and i+3 on the same helical face (i.e., the proline residues in the PXXP motifs, FIG. 1). Design of selective inhibitors for PXXP motif binding proteins, including Src, Abl, Nck, Crk, PIK3CA, RASp21, and Cdc25A, is of therapeutic interest. Additionally, the presence of extracellular PXXP motif binding domains affords an opportunity to develop peptide scaffolds as agonists or antagonists similar to conotoxin GLP-1 peptidomimetics or to engineered extendin-4 and PTH sequences. The entire content of each of References 1-8 is incorporated herein by reference.

Short peptide sequences covalently locked into discrete conformations have found utility for abrogating a wide variety of protein-protein interactions. For protein-protein interactions involving the binding of α-helices, there exists an arsenal of strategies for stapling side-chains together, or introducing hydrogen-bond surrogates to stabilize the helical secondary structure. A variety of stable (3-strand or hairpin structures, along with macrocyclic polypeptides including non-canonical amino acids, can be effective at modulating protein-protein interactions featuring (3-strands. In both of these modes of structural mimicry, hydrogen-bond networks play a critical role in templating the desired structure required for molecular recognition. However, protein-protein interactions are established via additional types of secondary structures, requiring new strategies to engender stability in structural mimetics of diverse peptide sequences present at protein-protein interfaces.

Proline is the only N-substituted canonical amino acid residue, making the design of sequences capable of achieving affinity and specificity for domains containing PXXP motif recognition elements difficult via design of canonical peptide sequences. Peptoids, or N-substituted glycine peptidomimetics, can effectively mimic the proline tertiary amide N-substitution, while also permitting incorporation of a wide array of synthetically tractable side-chain types. However, no synthetic methods exist to covalently constrain peptoid oligomers into discrete PPII-helical secondary structures. As described herein, the present inventors introduce a miniature protein scaffold that provides stability for PPII-helical elements comprising non-natural residues (e.g., peptoid residues). To this end, the present inventors sought to develop sequences containing a combination of peptoids and peptides in PPII-helical i and i+3 positions covalently locked by a disulfide-bridge. These hybrid structures establish a miniature protein platform to engineer sequences capable of achieving side-chain placements requisite for interrogating PXXP motif binding proteins of biomedical interest.

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

As described herein, the term “abiotic monomer” may be used to refer to an N-substituted glycine (also known as an N-substituted glycine peptoid or peptoid), an N-substituted amino acid (also described as a peptide tertiary amide), a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, or a urea-linked monomer.

Exemplary N-substituted glycine peptoids envisioned for incorporation into the miniature protein scaffolds described herein include any one of those described in U.S. Pat. No. 8,828,413; U.S. Application Publication Nos. 2014/0274916 and 2015/0044189; and Culf et al. (2010, Molecules 15:5282-5335), the entire content of each of which is incorporated herein by reference.

Exemplary N-Substituted Glycine Monomer Designators include the following:
Nap=N-(3-aminopropyl)glycine
Nab=N-(4-aminobutyl)glycine
Nah=N-(6-aminohexyl)glycine
Ngb=N-(4-guanidinobutyl)glycine
Npm=N-(phenylmethyl)glycine
Nnm=N-(naphthylmethyl)glycine
Ndp=N-(2,2-diphenylethyl)glycine
Nip=N-(isopropyl)glycine
Nib=N-(isobutyl)glycine

N-Substituted Glycine Monomer Designators:

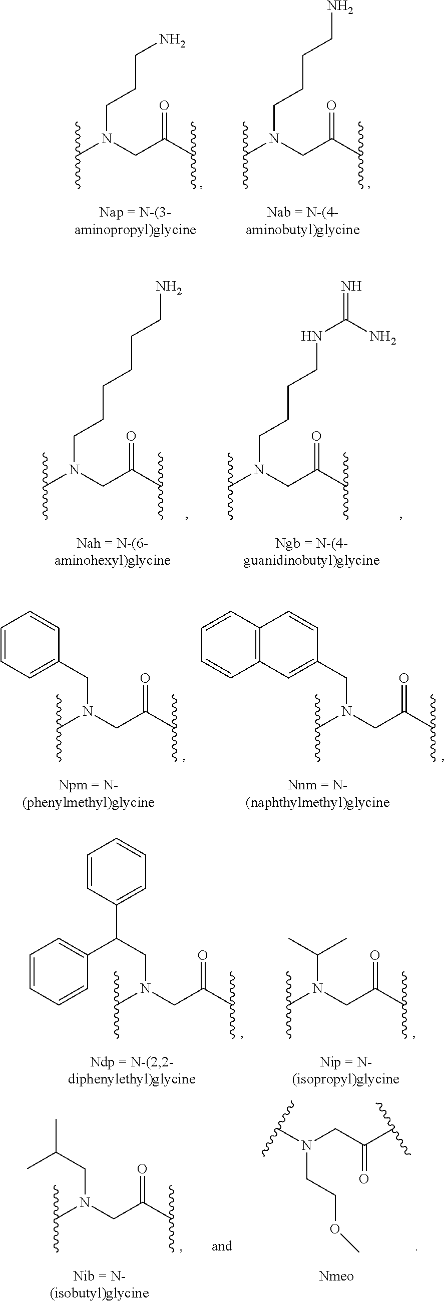

N-substituted amino acids are known in the art, as are methods for making same. See, for example, Pels et al. (2015, ACS Comb Sci 17:152-155), the entire content of which is incorporated herein by reference.

Monomers constituting oligoamide folded oligomers are known in the art and are described in, for example, Zhang et al. (2012, Chem Rev 112:5271-5316) and Guichard et al. (2011, Chem Commun 47:5933-5941), the entire content of each of which is incorporated herein by reference.

Side chain variants of Pro are known in the art and are described in, for example, Bach et al. (2013, Applied Microbiology and Biotechnology 97:6623-6634); Sigma Aldrich technical documents (available prior to Jun. 30, 2016) Chemfiles 5: Article 12, “Proline Derivatives and Analogs”; Anaspec Product catalog (available prior to Jun. 30, 2016), “Analogs of Proline”, the entire content of each of which is incorporated herein by reference.

‘Acyl’ or ‘Alkanoyl’ refers to a radical —C(O)R²⁰, where R²⁰ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein. Representative examples include, but are not limited to, formyl, acetyl, cyclohexylcarbonyl, cyclohexylmethylcarbonyl, benzoyl and benzylcarbonyl. Exemplary ‘acyl’ groups are —C(O)H, —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocycloalkyl), wherein t is an integer from 0 to 4.

‘Substituted Acyl’ or ‘Substituted Alkanoyl’ refers to a radical —C(O)R²¹, wherein R²¹ is independently
C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Alkoxy’ refers to the group —OR²⁹ where R²⁹ is C₁-C₈ alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

‘Substituted alkoxy’ refers to an alkoxy group substituted with one or more of those groups recited in the definition of “substituted” herein, and particularly refers to an alkoxy group having 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocycloalkyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups are —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are OCF₃, OCH₂CF₃, OCH₂Ph, OCH₂-cyclopropyl, OCH₂CH₂OH, and OCH₂CH₂NMe₂.

‘Alkyl’ means straight or branched aliphatic hydrocarbon having 1 to 20 carbon atoms. Particular alkyl has 1 to 12 carbon atoms. More particular is lower alkyl which has 1 to 6 carbon atoms. A further particular group has 1 to 4 carbon atoms. Exemplary straight chained groups include methyl, ethyl, n-propyl, and n-butyl. Branched means that one or more lower alkyl groups such as methyl, ethyl, propyl or butyl is attached to a linear alkyl chain, exemplary branched chain groups include isopropyl, iso-butyl, t-butyl and isoamyl.

‘Substituted alkyl’ refers to an alkyl group as defined above substituted with one or more of those groups recited in the definition of “substituted” herein, and particularly refers to an alkyl group having 1 or more substituents, for instance from 1 to 5 substituents, and particularly from 1 to 3 substituents, in particular 1 substituent, selected from the group consisting of acyl, acylamino, acyloxy (—O-acyl or —OC(O)R²⁰), alkoxy, alkoxycarbonyl, alkoxycarbonylamino (—NR^{″}-alkoxycarbonyl or —NH—C(O)—OR²⁷), amino, substituted amino, aminocarbonyl (carbamoyl or amido or —C(O)—NR^{″}₂), aminocarbonylamino (—NR^{″}—C(O)—NR^{″}₂), aminocarbonyloxy (—O—C(O)—NR^{″}₂), aminosulfonyl, sulfonylamino, aryl, aryloxy, azido, carboxyl, cyano, cycloalkyl, halogen, hydroxy, heteroaryl, nitro, thiol, —S-alkyl, —S-aryl, —S(O)-alkyl, —S(O)-aryl, —S(O)₂-alkyl, and —S(O)₂-aryl. In a particular embodiment ‘substituted alkyl’ refers to a C₁-C₈ alkyl group substituted with halo, cyano, nitro, trifluoromethyl, trifluoromethoxy, azido, —NR^{′″}SO₂R^{″}, —SO₂NR^{″}R^{′″}, —C(0)R″, -C(O)OR^{″}, —OC(O)R^{″}, —NR^{′″}C(O)R^{″}, —C(O)NR^{″}R^{′″}, —NR^{″}R^{′″}, or —(CR^{′″}R^{′″})ₘOR^{′″}; wherein each R″ is independently selected from H, C₁-C₈ alkyl, —(CH₂)ₜ(C₆-C₁₀ aryl), —(CH₂)ₜ(5-10 membered heteroaryl), —(CH₂)ₜ(C₃-C₁₀ cycloalkyl), and —(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Each of R^{′″} and R^{′″} independently represents H or C₁-C₈ alkyl.

‘Aralkyl’ or ‘arylalkyl’ refers to an alkyl group, as defined above, substituted with one or more aryl groups, as defined above. Particular aralkyl or arylalkyl groups are alkyl groups substituted with one aryl group.

‘Substituted Aralkyl’ or ‘substituted arylalkyl’ refers to an alkyl group, as defined above, substituted with one or more aryl groups; and at least one of the aryl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Aryl’ refers to a monovalent aromatic hydrocarbon group derived by the removal of one hydrogen atom from a single carbon atom of a parent aromatic ring system. In particular aryl refers to an aromatic ring structure, mono-cyclic or poly-cyclic that includes from 5 to 12 ring members, more usually 6 to 10. Where the aryl group is a monocyclic ring system it preferentially contains 6 carbon atoms. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene and trinaphthalene. Particularly aryl groups include phenyl, naphthyl, indenyl, and tetrahydronaphthyl.

‘Substituted Aryl’ refers to an aryl group substituted with one or more of those groups recited in the definition of ‘substituted’ herein, and particularly refers to an aryl group that may optionally be substituted with 1 or more substituents, for instance from 1 to 5 substituents, particularly 1 to 3 substituents, in particular 1 substituent. Particularly, ‘Substituted Aryl’ refers to an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

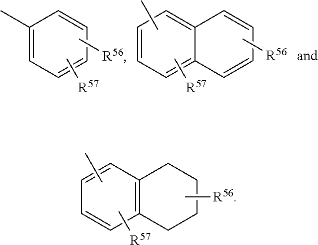

In these formulae one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocycloalkyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O or S. R⁶⁰ and R⁶′ are independently hydrogen, C1-C8 alkyl, C1-C4 haloalkyl, C3-C10 cycloalkyl, 4-10 membered heterocycloalkyl, C6-C10 aryl, substituted aryl, 5-10 membered heteroaryl.

‘Heteroaryl’ means an aromatic ring structure, mono-cyclic or polycyclic, that includes one or more heteroatoms and 5 to 12 ring members, more usually 5 to 10 ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Examples of five membered monocyclic heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups. Examples of six membered monocyclic heteroaryl groups include but are not limited to pyridine, pyrazine, pyridazine, pyrimidine and triazine. Particular examples of bicyclic heteroaryl groups containing a five membered ring fused to another five membered ring include but are not limited to imidazothiazole and imidazoimidazole. Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, isoindolone, indolizine, indoline, isoindoline, purine (e.g., adenine, guanine), indazole, pyrazolopyrimidine, triazolopyrimidine, benzodioxole and pyrazolopyridine groups. Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups. Particular heteroaryl groups are those derived from thiophene, pyrrole, benzothiophene, benzofuran, indole, pyridine, quinoline, imidazole, oxazole and pyrazine.

Examples of representative heteroaryls include the following:

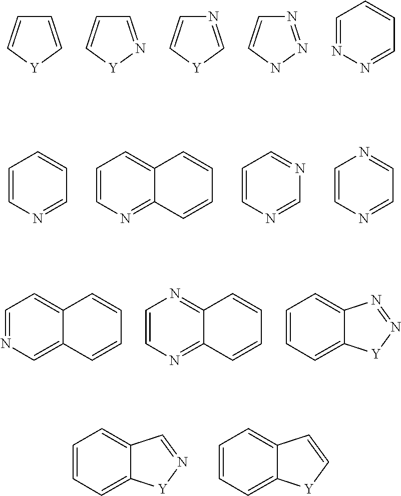

wherein each Y is selected from carbonyl, N, NR⁶⁵, O and S; and R⁶⁵ is independently hydrogen, C₁-C₈ alkyl, C₃-C₁₀ cycloalkyl, 4-10 membered heterocycloalkyl, C₆-C₁₀ aryl, and 5-10 membered heteroaryl.

Examples of representative aryl having hetero atoms containing substitution include the following:

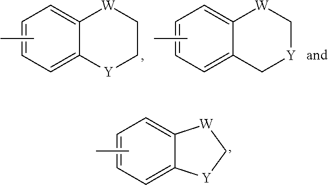

wherein each W is selected from C(R⁶⁶)₂, NR⁶⁶, O and S; and each Y is selected from carbonyl, NR⁶⁶, O and S; and R⁶⁶ is independently hydrogen, C₁-C₈ alkyl, C₃-C₁₀ cycloalkyl, 4-10 membered heterocycloalkyl, C₆-C₁₀ aryl, and 5-10 membered heteroaryl.

The terms “abiotic monomers”, “non-natural residues”, or “unnatural amino acids” are used to refer to, for example, N-substituted glycine residues (all the variations of N-substituted glycine), N-substituted amino acids (such as, for example, N-methyl alanine, N-methyl phenylalanine), and side chain variations of proline (for example, 4-fluoro-proline. Such abiotic monomers, non-natural residues, or unnatural amino acids may be prepared and used individually in accordance with the present invention, or may incorporated into existing proteins. A general method for site-specific incorporation of unnatural amino acids into proteins is described in Christopher J. Noren, Spencer J. Anthony-Cahill, Michael C. Griffith, Peter G. Schultz, Science, 244:182-188 (April 1989).

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject not yet exposed to or predisposed to the disease,and not yet experiencing or displaying symptoms of the disease).

“Prodrugs” refers to compounds, including derivatives of the compounds provided herein,which have cleavable groups and become by solvolysis or under physiological conditions the compounds provided herein which are pharmaceutically active in vivo. Such examples include, but are not limited to, choline ester derivatives and the like, N-alkylmorpholine esters and the like.

“Solvate” refers to forms of the compound that are associated with a solvent, usually by a solvolysis reaction. Conventional solvents include water, ethanol, acetic acid and the like. The compounds provided herein may be prepared e.g. in crystalline form and may be solvated or hydrated. Suitable solvates include pharmaceutically acceptable solvates, such as hydrates, and further include both stoichiometric solvates and non-stoichiometric solvates.

“Subject” includes humans. The terms “patient” and “subject” are used interchangeably herein. Accordingly, a subject can be a mammal, in a particular embodiment a human, or a bird, a reptile, an amphibian, or a plant.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both.

Other derivatives of the compounds provided herein have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds provided herein are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds provided herein.

As used herein, the term “isotopic variant” refers to a compound that comprises an unnatural proportion of an isotope of one or more of the atoms that constitute such compound. For example, an “isotopic variant” of a compound can comprise an unnatural proportion of one or more non-radioactive isotopes, such as for example, deuterium (²H or D), carbon-13 (¹³C), nitrogen-15 (¹⁵N), or the like. It will be understood that, in a compound comprising an unnatural proportion of an isotope, any example of an atom where present, may vary in isotope composition. For example, any hydrogen may be ²H/D, or any carbon may be ¹³C, or any nitrogen may be ¹⁵N, and that the presence and placement of such atoms may be determined within the skill of the art. Likewise, provided herein are methods for preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸ F, ¹⁵O and ¹³N, and would be useful in Positron Emission Topography (PET) studies for examining substrate receptor occupancy. All isotopic variants of the compounds provided herein, radioactive or not, are intended to be encompassed within the scope provided herein.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

### Protein Scaffolds

As set forth earlier herein, the miniature synthetic protein scaffolds described herein comprise PPII-helical and/or beta-strand peptidomimetics. Accordingly, these compounds (i.e., the miniature synthetic protein scaffolds) may be used as selective inhibitors for PXXP and beta-strand motif binding proteins and protein-protein interactions dependent on same. As described herein, these miniature synthetic protein scaffolds may comprise abiotic monomers, such as, for example, peptoids, beta-amino acids, D-amino acids, and/or non-indigenous amino acids.

Peptoids exhibit many advantageous characteristics for development of bioactive compounds, as they are amenable to efficient solid phase synthesis; can incorporate highly diverse chemical functionalities; can establish a relationship between oligomer sequence, three-dimensional structure, and function; do not require the presence of chiral centers; can demonstrate marked resistance to degradation; have superior cell permeability characteristics relative to peptides; and can manifest rapid bioactivities. Some of the advantageous properties of peptidomimetics for use as, for example, antibiotics are described in Srinivas et al. (Science 2010, 327, 1010-1013).

More particularly, the present invention relates to synthetic PPII-helical/beta-strand scaffolds (also referred to herein as miniature protein scaffolds or synthetic miniature protein scaffolds) that serve as peptidomimetics for PPII-helices and/or beta-strands, as described herein below.

A miniature protein scaffold comprising Arg Val Arg Val Arg Thr Ser Arg Xaa Gly Tyr Asn Gly Thr Trp Ser Glu Trp Ser, wherein Xaa at position 9 is proline (Pro) or D-Pro and at least one of the arginine (Arg) at position 1, 4, or 8; the valine (Val) at position 2 or 4; the threonine (Thr) at position 6 or 14; the serine (Ser) at position 7, 16, or 19; the tyrosine (Tyr) at position 11; the asparagine (Asn) at position 12; the glycine (Gly) at position 13; and the glutamic acid (Glu) at position 17; is replaced by an abiotic monomer, provided that the abiotic monomer is other than D-Pro or a substituted Pro residue.

A miniature protein scaffold comprising Arg Cys Arg Val Arg Thr Ser Arg D-Pro Gly Tyr Asn Gly Thr Trp Ser Glu Trp Cys, wherein at least one of the arginine (Arg) at position 1, 4, or 8; the valine (Val) at position 4; the threonine (Thr) at position 6 or 14; the serine (Ser) at position 7 or 16; the tyrosine (Tyr) at position 11; the asparagine (Asn) at position 12; the glycine (Gly) at position 13; and the glutamic acid (Glu) at position 17; is replaced by an abiotic monomer, provided that the abiotic monomer is other than D-Pro or a substituted Pro residue. In a particular embodiment thereof, the cysteines (Cys) at positions 2 and 19 are linked via a disulfide bridge.

The miniature protein scaffold comprises at least one abiotic monomer is selected from an N-substituted glycine (also known as an N-substituted glycine peptoid or peptoid), an N-substituted amino acid (also described as a peptide tertiary amide), a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

In accordance with the discoveries of the present inventors, the miniature protein scaffold comprising at least one abiotic monomer may comprise at least one and up to fourteen abiotic monomers. Accordingly, the miniature protein scaffold may comprise 1, 2, 3, 4, 5, 6, 7, 8, 9, 10, 11, 12, 13, or 14 abiotic monomers.

In one embodiment the abiotic monomer is a peptoid monomer having the following structure:

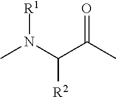

wherein a peptoid monomer may be according to formula IIa, IIb, IIc, or IId:

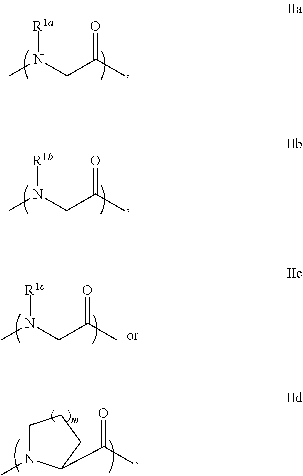

wherein

each R^{l} is independently substituted or unsubstituted alkyl;

each R² is independently hydrogen, or substituted or unsubstituted alkyl; or each R¹ and R² are join together to form a 4-7 membered heterocyclic ring;

each R^{1a} is independently unsubstituted alkyl;

each R^{1b} is independently aminoalkyl, guanidinoalkyl (H₂N—C(═NH)—NH-alkyl), or N-containing heteroarylalkyl;

each R^{1c} is independently substituted or unsubstituted aryl, substituted or unsubstituted aralkyl, substituted or unsubstituted heteroarylalkyl,or substituted or unsubstituted diarylalkyl;

each m is 0, 1, 2, or 3;

or a salt thereof; and stereoisomers, isotopic variants and tautomers thereof

In one embodiment, with respect to protein scaffolds described herein, the abiotic monomer is a peptoid wherein R^{1a} is unsubstituted alkyl.

In one embodiment, with respect to protein scaffolds described herein, the abiotic monomer is a peptoid wherein R^{1a} is alkyl, substituted with halo, hydroxy, amino, nitro, and alkoxy.

In another embodiment, with respect to protein scaffolds described herein, the abiotic monomer is a peptoid wherein R^{1a} is alkyl, substituted with halo. In one particular embodiment, R^{1a} is alkyl, substituted with F. In another particular embodiment, R^{1a} is alkyl, substituted with one or more F.

In one embodiment, with respect to protein scaffolds described herein, the abiotic monomer is a peptoid wherein R^{1a} is Me, Et, n-Pr, i-Pr, n-Bu, sec-Bu, or i-Bu.

In one embodiment, with respect to protein scaffolds described herein, the abiotic monomer is a peptoid wherein R^{1c} is benzyl, unsubstituted or substituted with one or more groups selected from alkyl, halo, hydroxy, amino, nitro, and alkoxy.

In one embodiment, with respect to protein scaffolds described herein, the abiotic monomer is a peptoid wherein R^{1c} is phenyl, unsubstituted or substituted with one or more groups selected from alkyl, halo, hydroxy, amino, nitro, and alkoxy.

In one embodiment, with respect to protein scaffolds described herein, the abiotic monomer is a peptoid wherein R^{1c} is phenyl, substituted with one or more halo. In one particular embodiment, with respect to protein scaffolds described herein, the abiotic monomer is a peptoid wherein R^{1c} is phenyl, substituted with one or more F.

In one embodiment, with respect to protein scaffolds described herein, the abiotic monomer is a peptoid wherein R^{1c} is unsubstituted phenyl.

In one embodiment, with respect to protein scaffolds described herein, the abiotic monomer is a peptoid wherein R^{1c} is naphthyl, unsubstituted or substituted with one or more groups selected from alkyl, halo, hydroxy, amino, nitro, and alkoxy.

In one embodiment, with respect to protein scaffolds described herein, the abiotic monomer is a peptoid wherein R^{1c} is unsubstituted naphthyl.

In one embodiment, with respect to protein scaffolds described herein, the abiotic monomer is a peptoid wherein R^{1c} is naphthyl, substituted with one or more halo. In one particular embodiment, with respect to protein scaffolds described herein, the abiotic monomer is a peptoid wherein R^{1c} is naphathyl, substituted with one or more F.

In one embodiment, with respect to protein scaffolds described herein, the abiotic monomer is a peptoid wherein R^{1c} is phenethyl, unsubstituted or substituted with one or more groups selected from alkyl, halo, hydroxy, amino, nitro, and alkoxy.

In one embodiment, with respect to protein scaffolds described herein, the abiotic monomer is a peptoid wherein R^{1c} is phenethyl, substituted with one or more halo. In one particular embodiment, with respect to protein scaffolds described herein, the abiotic monomer is a peptoid wherein R^{1c} is phenethyl, substituted with one or more F.

In one embodiment, with respect to protein scaffolds described herein, the abiotic monomer is a peptoid wherein R^{1c} is 2-naphthyl, unsubstituted or substituted with one or more groups selected from alkyl, halo, hydroxy, amino, nitro, and alkoxy. In one particular embodiment, the abiotic monomer is a peptoid wherein R^{1c} is 2-naphthyl, substituted with one or more F.

In one embodiment, with respect to protein scaffolds described herein, the abiotic monomer is a peptoid wherein R^{1c} is 2,2-diphenylethyl, unsubstituted or substituted with one or more groups selected from alkyl, halo, hydroxy, amino, nitro, and alkoxy. In one particular embodiment, the abiotic monomer is a peptoid wherein R^{1c} is 2,2-diphenylethyl, substituted with one or more F.

In one embodiment, with respect to protein scaffolds described herein, the abiotic monomer is a peptoid wherein R^{1c} is furanyl or thienyl, unsubstituted or substituted with one or more groups selected from alkyl, halo, hydroxy, amino, nitro, and alkoxy. In one particular embodiment, the abiotic monomer is a peptoid wherein R^{1c} is furanyl or thienyl, substituted with one or more F.

In one embodiment, with respect to protein scaffolds described herein, m is 1 or 2.

In one embodiment, with respect to protein scaffolds of formula I, m is 1.

In one embodiment, with respect to protein scaffolds described herein, the abiotic monomer is a peptoid wherein R^{1b} is aminomethyl, 2-aminoethyl, 3-aminopropyl, 4-aminobutyl, 5-aminopentyl, or 6-aminohexyl.

In one embodiment, with respect to protein scaffolds described herein, the abiotic monomer is a peptoid wherein R^{1b} is aminomethyl, 2-aminoethyl, 3-aminopropyl, 4-aminobutyl, 5-aminopentyl, or 6-aminohexyl; and wherein the aminoalkyl groups (aminomethyl, 2-aminoethyl, 3-aminopropyl, 4-aminobutyl, 5-aminopentyl, or 6-aminohexyl) may be substituted with one or more F.

In one embodiment, with respect to protein scaffolds described herein, the abiotic monomer is a peptoid wherein R^{1b} is 3-aminopropyl.

In one embodiment, with respect to protein scaffolds described herein, the abiotic monomer is a peptoid wherein R^{1b} is guanidinoalkyl.

In one embodiment, with respect to protein scaffolds described herein, the abiotic monomer is a peptoid wherein R^{1b} is guanidinomethyl, 2-guanidinoethyl, 3-guanidinopropyl, 4-guanidinobutyl, 5-guanidinopentyl, or 6-guanidinohexyl.

In one embodiment, with respect to protein scaffolds described herein, the abiotic monomer is a peptoid wherein R^{1b} is guanidinomethyl, 2-guanidinoethyl, 3-guanidinopropyl, 4-guanidinobutyl, 5-guanidinopentyl, or 6-guanidinohexyl; and wherein the guanidinoalkyl groups may be substituted with one or more F.

In one embodiment, with respect to protein scaffolds described herein, the abiotic monomer is a peptoid wherein R^{1b} is 4-guanidinobutyl.

In one embodiment, with respect to protein scaffolds described herein, the abiotic monomer is a peptoid wherein R^{1b} is imidazolylmethyl, 2-imidazolylethyl, 3-imidazolylpropyl, 4-imidazolylbutyl, 5-imidazolylpentyl, or 6-imidazolylhexyl.

In one embodiment, with respect to protein scaffolds described herein, the abiotic monomer is a peptoid wherein R^{1b} is imidazolylmethyl, 2-imidazolylethyl, 3-imidazolylpropyl, 4-imidazolylbutyl, 5-imidazolylpentyl, or 6-imidazolylhexyl; and wherein the imidazoalkyl groups may be substituted with one or more F

In one embodiment, with respect to protein scaffolds described herein, the abiotic monomer is a peptoid wherein R^{1b} is methyl, n-propyl, n-butyl, n-pentyl, and n-hexyl, substituted with pyrrolyl, oxazolyl, isoxazolyl, thiazolyl, thiadiazolyl, triazolyl, or tetrazolyl.

In one embodiment, with respect to protein scaffolds described herein, the abiotic monomer is a peptoid wherein R^{1b} is methyl, n-propyl, n-butyl, n-pentyl, and n-hexyl, substituted with pyrrolyl, oxazolyl, isoxazolyl, thiazolyl, thiadiazolyl, triazolyl, or tetrazolyl; and all these groups are substituted with one or more F.

In a particular embodiment thereof, the abiotic monomer is selected from the group consisting of an N-substituted glycine, an N-substituted amino acid, or a side chain variant of Pro.

In one embodiment, with respect to miniature protein scaffolds described herein, the peptoid monomer is selected from Nap, Nab, Nah, Ngb, Npm, Nnm, Ndp, Nip, Nib, and Nmeo:

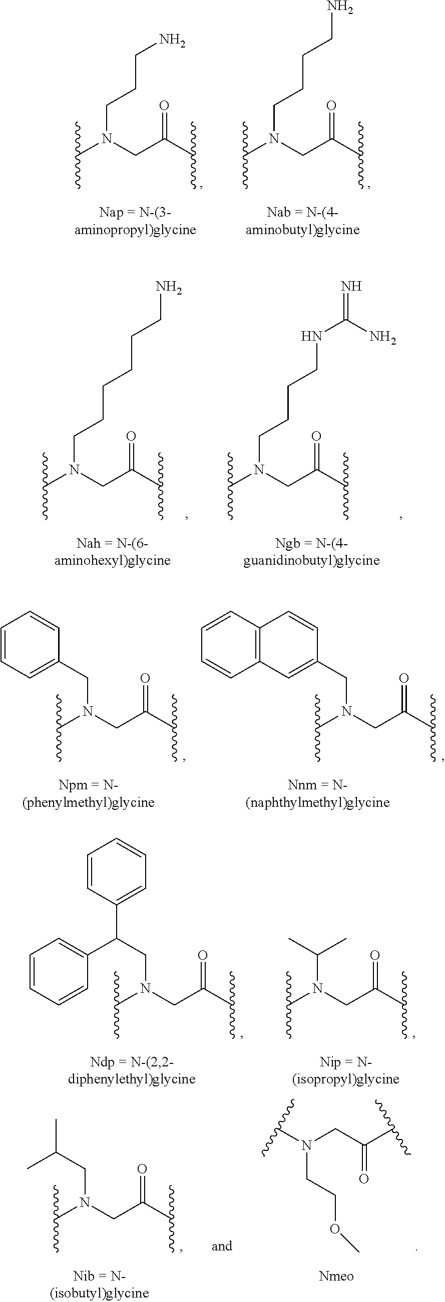

In one embodiment, with respect to miniature protein scaffolds described herein, the peptoid monomer or N-substituted glycine is selected from the group consisting of N-(methoxyethyl) glycine, N—(S/R)-phenylethylamine glycine, N-(aryl) glycine, and N-(alkyl) glycine.

In certain aspects and where appropriate, the present invention extends to the preparation of prodrugs and derivatives of the protein scaffolds of the invention. Prodrugs are derivatives which have cleavable groups and become by solvolysis or under physiological conditions the peptoid of the invention, which are pharmaceutically active, in vivo.

In one embodiment, the invention provides a pharmaceutical composition comprising a pharmaceutically acceptable carrier and a pharmaceutically effective amount of a protein scaffold described herein.

In one embodiment, the invention provides a pharmaceutical composition of a protein scaffold described herein, comprising a pharmaceutically acceptable carrier, and the carrier is a parenteral carrier, oral or topical carrier.

In one embodiment, the invention provides a method for preventing, treating, ameliorating or managing a disease or condition which comprises administering to a patient in need of such prevention, treatment, amelioration or management, a prophylactically or therapeutically effective amount of the pharmaceutical composition of a protein scaffold described herein.

In one embodiment, the disease, disorder, or condition is related to or associated with protein-protein interactions directly mediated by poly-proline type-II helix and/or beta-strand interactions.

In one embodiment, the disease, disorder, or condition is related to or associated with protein-protein interactions mediated by poly-proline type-II helix domains of Src, Abl, Nck, Crk, PIK3CA, RASp21, or Cdc25A.

In one embodiment, the invention provides a method for preventing, treating, ameliorating or managing a disease or condition, which comprises administering to a subject in need of such prevention, treatment, amelioration or management a prophylactically or therapeutically acceptable amount of a protein scaffold described herein, or a pharmaceutical composition thereof, wherein the disease, disorder, or condition is, for example, a cancer, muscular dystrophy, Alzheimer and Huntington diseases, Liddle's syndrome of hypertension, and a number of X chromosome linked intellectual disabilities. See, for example, Pucheta-Martinez et al. (2016, Science Reports 6:30293), the entire content of which is incorporated herein by reference.

As discussed in, for example, Zarrinpar et al. (2003, Science STKE 179:RE8; the entire content of which is incorporated herein by reference), proline recognition domains are domains that are typically present in multidomain signaling proteins that comprise other signaling/recognition domains. Binding events of such proteins contribute to a wide variety of cellular functions including the assembly and targeting of protein complexes involved in cell growth, cytoskeletal rearrangements, transcription, postsynaptic signaling, and other key cellular processes. Accordingly, it will be appreciated that proline recognition domains such as SH3 and WW domains are thought to contribute to these cellular functions and diseases associated therewith. Accordingly, miniature protein scaffolds and compositions thereof described herein are envisioned as therapeutics having utility in methods for treating diseases associated with proteins comprising proline recognition domains.

### Pharmaceutical Compositions

When employed as pharmaceuticals, the protein scaffold compounds of this invention are typically administered in the form of a pharmaceutical composition. Such compositions can be prepared in a manner well known in the pharmaceutical art and comprise at least one active complex. In a further embodiment, the pharmaceutical compositions of the invention may comprise one or more of the protein scaffold compounds in combination with other compounds that are useful for the treatment of the aforementioned conditions. Such combinations yield compositions that exhibit improved effectiveness over like compositions containing the active compounds individually, so that a synergistic effect of the combination is conferred. The exact amounts and proportions of the compounds with respect to each other may vary within the skill of the art.

Generally, the protein scaffold compound of this invention is administered in a pharmaceutically effective amount. The amount of the complex actually administered will typically be determined by a physician, in the light of the relevant circumstances, including the condition to be treated, the chosen route of administration, the actual complex administered, the age, weight, and response of the individual patient, the severity of the patient's symptoms, and the like.

The pharmaceutical compositions of this invention can be administered by a variety of routes including by way of non limiting example, oral, rectal, vaginal, transdermal, subcutaneous, intravenous, intramuscular and intranasal. Depending upon the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention may be admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2—Capsules

A compound of the invention may be admixed as a dry powder with a starch diluent in an approximate 1:1 weight ratio. The mixture is filled into 250 mg capsules (125 mg of active compound per capsule).

### Formulation 3—Liquid

A compound of the invention (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then be added to produce a total volume of 5 mL.

### Formulation 4—Tablets

A compound of the invention may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5—Injection

A compound of the invention may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) may be added and the resulting mixture is stirred until it congeals.

### Methods of Treatment

The present protein scaffold compounds and compositions thereof may be used as therapeutic agents for the treatment of conditions in mammals. Accordingly, the compounds and pharmaceutical compositions of this invention find use as therapeutics for preventing and/or treating a disease, disorder, or condition related to or associated with protein-protein interactions directly mediated by poly-proline type-II helix interactions in mammals, including humans. Exemplary proteins comprising poly-proline type-II helix domains include, without limitation, Src, Abl, Nck, Crk, PIK3CA, RASp21, or Cdc25A. Thus, and as stated earlier, the present invention includes within its scope, and extends to, the recited methods of treatment, as well as to the compounds for such methods, and to the use of such compounds for the preparation of medicaments useful for such methods.

In a method of treatment aspect, this invention provides a method of treating a mammal susceptible to or afflicted with a disease, disorder, or condition related to or associated with protein-protein interactions directly mediated by poly-proline type-II helix interactions, which method comprises administering an effective amount of one or more of the pharmaceutical compositions just described.

The methods disclosed herein have veterinary applications and can be used to treat a wide variety of non-human vertebrates. Thus, in other aspects of the invention, the compositions of the present invention are administered in the above methods to non-human vertebrates, such as wild, domestic, or farm animals, including, but not limited to, cattle, sheep, goats, pigs, dogs, cats, and poultry such as chicken, turkeys, quail, pigeons, ornamental birds and the like.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound or its derivative, with preferred doses each providing from about 0.1 to about 10 mg/kg and especially about 1 to about 5 mg/kg.

Transdermal doses are generally selected to provide similar or lower blood levels than are achieved using injection doses.

The protein scaffold compounds of this invention can be administered as the sole active agent or they can be administered in combination with other agents, including other active derivatives.

A skilled practitioner would appreciate that the choice as to which compound or compounds of the invention are well suited to a particular application must take into consideration such variables as the severity of the disease or condition, mode of administration, and duration of administration.

### General Synthetic Procedures

The compounds of this invention can be prepared from readily available starting materials using the general methods and procedures described earlier and illustrated schematically in the examples that follow and in references cited therein. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of representative protein scaffolds that have been listed herein. The protein scaffolds of the invention may be prepared from known or commercially available starting materials and reagents by one skilled in the art of organic synthesis. See, for example, Craven et al. (2016, Chem. Soc. 138:1543-1550), the entire content of which is incorporated herein by reference.

### EXAMPLES

The following examples are presented in order to more fully illustrate the preferred embodiments of the invention. They should in no way be construed, however, as limiting the broad scope of the invention.

### Example 1

The TrpPlexus miniature protein as a platform for the design of peptoid-peptide hybrid PXXP motif mimetics. The present inventors recently demonstrated^{[18]} the folding of a proline-free PPII-helix in the context of a miniature protein stabilized by a network of cation-n interactions. These interactions include an intercalated network of tryptophan indole (Trp15, Trp18) and arginine guanidinium (Arg1, Arg3, and Argy) side-chain groups (FIG. 1). This 19-residue “TrpPlexus” protein structure also included two solvent-exposed residues (i and i+3: Thr14 and Glu17, FIG. 1A) with poor PPII-helical propensities in a proline-free secondary structural mimetic of a PXXP motif. TrpPlexus was found to have a ΔG^{o}_{folding} of approximately −6.7 kcal/mol. The energetic preference of the sequence to fold into a cation-n interaction mediated tertiary structure is predicated upon presenting PPII-helical co-facial tryptophan residues (i and i+3: Trp15 and Trp18) capable of intercalating arginine residues. The cation-π interactions provided a driving force sufficient to enforce a PPII-helical conformation at the neighboring Thr14 and Glu17 residues. This finding suggested that non-covalent interactions could similarly enforce PPII-helices for a variety of additional residue types at positions 14 and 17. The present inventors sought to evaluate which combinations of residue types (i and i+3: positions 14 and 17) could access the PPII-helical conformation required to achieve the cation-π interaction network found in the TrpPlexus fold.

The cyclic TrpPlexus miniature protein. The installation of cyclic constraints into polypeptides has the potential to collapse a multi-conformational ensemble into a discrete and well-defined structure. As part of the initial report of the TrpPlexus miniature protein, the present inventors also engineered a disulfide-bridged version. This cyclic constraint could allow for structuring of residue types such as peptoids, which typically lack a single conformation. Peptoids are typically iso-energetic for several combinations of φ, ψ, and amide co backbone dihedral angles. The design of peptoid sequences having an energetic preference for PPI or PPII secondary structures has been limited to specific side-chain types. A covalently constrained peptoid-peptide hybrid miniature protein scaffold would greatly benefit peptoid design efforts by allowing for a much broader array of side-chain types to be locked into PPII-helices.

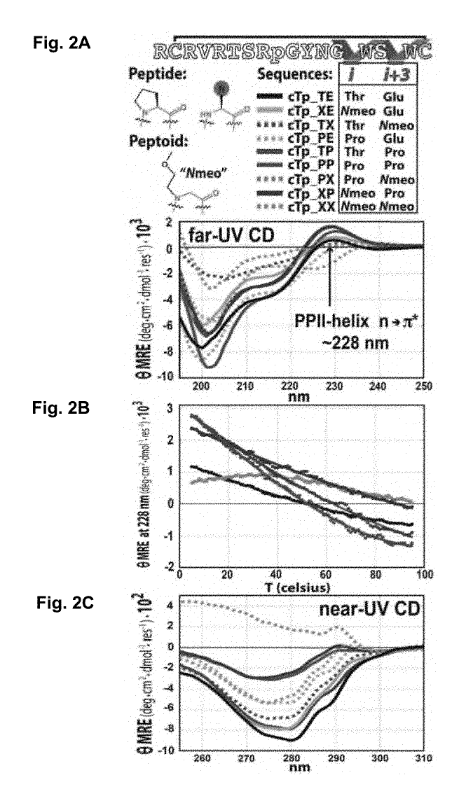

Design of PXXP mimetic TrpPlexus sequences. The present inventors designed versions of the cyclic TrpPlexus miniature protein with peptoid and proline residues replacing the solvent-exposed Thr14 (i) and Glu17 (i+3) residues within the PPII-helix (FIG. 2). The N-(methoxyethyl)-glycine or “Nmeo” monomer was selected as a representative peptoid residue as it exhibits the typical physico-chemical features of common peptoid side-chain types. This peptoid residue is also iso-energetic for several combinations of φ, ψ, as well as cis and trans w backbone dihedral angles. Sequence variants including proline residues were also included due to the fact that this is the native residue generally required for PXXP motif recognition. Proteins that include a combination of proline and peptoid residues may be desirable sequences for a variety of design objectives. Previous studies have illustrated the conformational features disfavoring PPII-helices for proline residues adjacent to large aromatic groups. Therefore, in select cases such as the TrpPlexus fold, proline compatibility with the PPII-helix is not guaranteed.

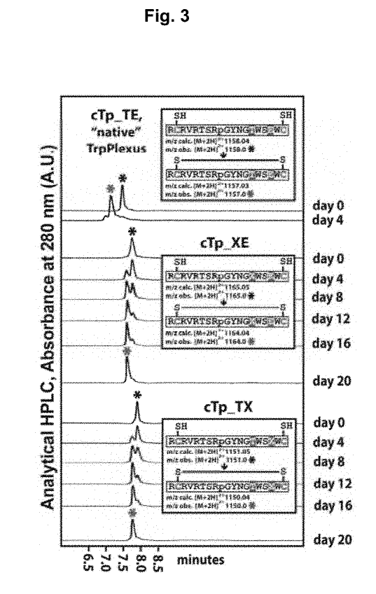

Synthesis and characterization of PXXP mimetic TrpPlexus sequences. The nine cyclic miniature protein sequences (listed in FIG. 2) were synthesized in an analogous manner to previously published protocols. The entire content of Reference 18 is incorporated herein by reference. Disulfide-bridge formation was monitored via analytical HPLC and ESI-mass spectrometry to observe the change in HPLC retention time and loss of sulfhydryl proton masses upon cyclization (FIG. 3). All compounds were found to cyclize and elute in a single peak by analytical HPLC.

Secondary structure content of each of the cyclized sequences was evaluated by circular dichroism (CD) spectroscopy. CD spectra were collected in the far-UV (195 nm to 250 nm) region to determine if sequences have features indicative of PPII-helical secondary structures, including a positive lobe of ellipticity at 228 nm characteristic of PPII-helical amide n→πr*transitions. Several spectra, including those for sequences containing peptoid residues, were found to display prominent features indicating the presence of a PPII-helix as well as matching the far-UV CD spectrum for TrpPlexus. Interestingly, a sequence containing a peptoid mutation at position 14 (cTp_XE) appears to be PPII-helical whereas the corresponding proline mutant (cTp_PE) did not. In contrast, for mutants at position 17, proline is the only residue resulting in a PPII-helical far-UV CD signature. Despite the observation that a single proline residue mutation at position 14 was not conducive to forming a PPII-helical structure, the double proline mutant (cTp_PP) appears to achieve this conformation.

The thermal stability of sequences containing PPII-helical far-UV CD features was ascertained from the temperature dependence of the ellipticity at 228 nm (FIG. 2B). The trends in ellipticity vs. temperature generally agreed with those observed for proline-rich PPII-helices, specifically that the mean residue ellipticity (MRE) values decrease as a function of increasing temperature. There is continuing debate on how to develop metrics to quantify the fraction of folded PPII-helix in protein structures by far-UV CD measurements, as MRE values for folded and unfolded structures are frequently dependent on the types of residues found in the PPII-helical regions. Nevertheless, the present inventors conclude that the thermo-stabilities of PPII-helices in structures featuring peptoid and proline mutants are similar. Notably, incorporation of peptoid residues in two sequences (cTp_XE and cTp_XP) did not diminish the stability of PPII-helical structure, as both mutation sets appeared to slightly enhance the thermal stability of the helical positions.

Near-UV CD spectra (255 nm to 310 nm) were obtained in order to evaluate the tertiary structure content of each sequence. The near-UV CD spectra arise from the ¹Lₐ, and ¹L_{b} transitions of tryptophan and tyrosine side-chains positioned in the asymmetric environments established by the formation of tertiary contacts. The spectra for several sequences indicated the presence of tertiary structure comparable to the published TrpPlexus sequence, as well as alternate combinations of tryptophan side-chain rotamers compatible with the cation-n interaction stabilized tertiary structure.

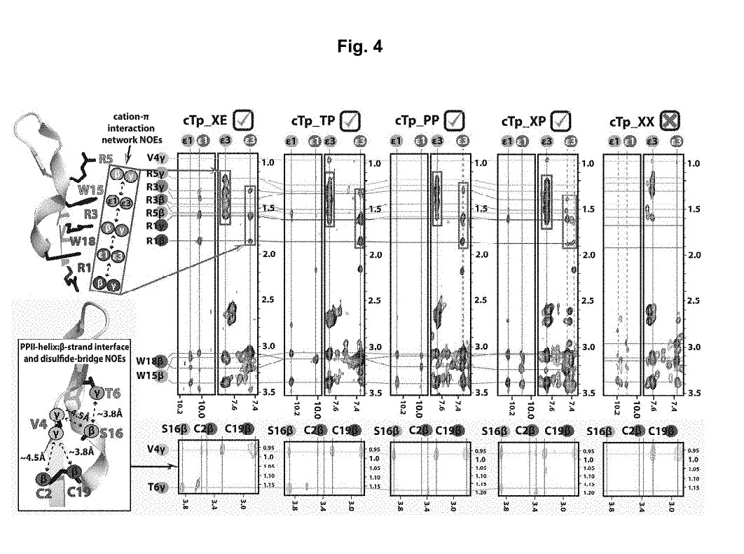

To gain a better understanding of the 3-dimensional structure of the cyclized proteins, the present inventors conducted NMR experiments. Peptide side-chain chemical shifts were assigned based upon a combination of 2D ¹H—¹H TOCSY and NOESY spectra. The critical NOEs characteristic of the cation-n interaction network include those indicating intercalation of the tryptophan indole groups between the arginine side-chain protons (FIG. 4). The NOESY spectra for several proteins featured strong NOEs between the ε1,ε3 indole ring atoms of Trp15/Trp18 and the β,γ side-chain atoms of Arg1/Arg3/Arg5. The presence of the cation-n interaction network was observed in two sequences containing peptoid residues (cTp_XE and cTp_XP). Additionally, several NOEs confirming the desired geometry of the interface between the PPII-helix and adjacent strand secondary structural elements, as well as the disulfide-bridge structure, were observed for these sequences (FIG. 4). The ¹H—¹H NOESY spectrum for a sequence determined to be unfolded (cTp_XX) can be seen in FIG. 4 in comparison to the spectra of other folded sequences. The disulfide-bridge was confirmed for all sequences from the presence of strong Val4γ→Cys19β NOEs (FIG. 4).

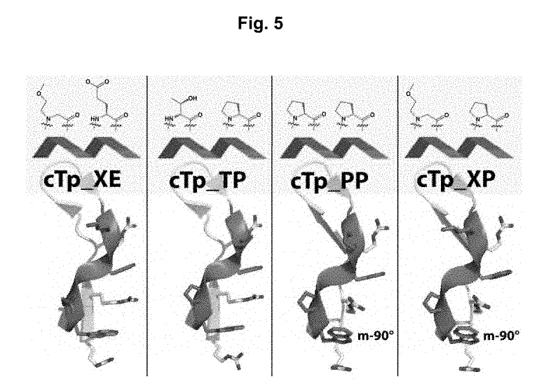

Using a combination of CD and NMR spectral features, the present inventors confirmed that four combinations of peptoid and peptide residues at i and i+3 positions form a PPII-helix in the cyclic TrpPlexus fold. These residue combinations and (i and i+3: Nmeo-Glu, Thr-Pro, Pro-Pro, and Nmeo-Pro, FIGS. 4 and 5) were found to match both observed CD and NOEs reported for the TrpPlexus sequence^{[18]} with only small differences. Primarily, the Trp18 rotamers in sequences cTp_PP and cTp_XP were found to be different than the m95° rotamer. For these sequences, the m-90° is the most probable rotamer (seen modeled in FIG. 5) determined from a combinaton of NOEs as well as near-UV CD spectra (see supporting information for additonal details).

Finally, the present inventors generated structural models in agreement with both observed long-range unambiguous NOEs as well as near and far-UV CD spectra. For sequences confirmed to be folded, each of the cyclic TrpPlexus variants was modeled in the Rosetta Molecular Design Package. The sequences all exhibit the identical cation-π interaction network and disulfide-bridge as the NMR solution structure of the TrpPlexus fold (FIG. 1B). The calculated minimum energy arginine rotamers are presented at positions Arg1, Arg3, and Arg5. However, these side-chains are most likely in active flux between several rotamers each being individually capable of maintaining the cation-π interaction network. Notably, in each of these variant sequences, a cation-n interaction network provides a sufficient energetic driving force to achieve a β:loop:PPII-helix tertiary structure with minimal side-chain and backbone perturbations (FIG. 5).

In summary, the design, synthesis, and characterization of a miniature protein scaffold is disclosed, that is capable of presenting several combinations of peptide and peptoid residues at the cofacial i and i+3 positions of a PPII-helix (FIG. 5). These findings validate a strategy for achieving protein-mimetic tertiary structures constituted from peptide and abiotic “foldamer” units. The ability to incorporate diverse monomer types within the PPII-helical motif provides a new avenue for discovery of peptide and peptidomimetic hybrids that can target PXXP motif-binding proteins and potentially modulate cellular signaling processes.

From the foregoing description, various modifications and changes in the compositions and methods of this invention will occur to those skilled in the art. All such modifications coming within the scope of the appended claims are intended to be included therein.

It is further understood that amino acid sizes, and all molecular weight or molecular mass values, given for synthetic protein scaffolds described herein are approximate, and are provided for description.

All publications, including but not limited to patents and patent applications, cited in this specification are herein incorporated by reference as if each individual publication were specifically and individually indicated to be incorporated by reference herein as though fully set forth.

### REFERENCES

[1] A. A. Adzhubei, M. J. Sternberg, A. A. Makarovi J. Mol. Biol. 2013 425, 2100-2132.

[2] R. Opitz, M. Muller, C. Reuter, M. Barone, A. Soicke, Y. Roskec, K. Piotukha, P. Huy, M. Beerbaum, B. Wiesner, M. Beyer man P.Schmieder, C. friend, R.Volkmer, H. Oschkinat, H.-G. Schmalz, R. Kühne Proc. Natl. Acad. Sci. U.S.A. 2015, 112, 5011-5016.

[3] P. J. Mintz, M. Cardo-Vila, M. G. Ozawa, A. Hajitou, R. Rangel, et al. Proc. Natl. Acad. Sci. U.S.A. 2009, 106, 2182-2187.

[4] R. Stoll, C. Renner, M. Zweckstetter, M. Bruggert, D. Ambrosius, S. Palme, R. A. Engh, M. Golob, I. Breibach, R. Buettner, W. Voelter, T. A. Holak, A. K. Bosserhoff EMBO 1 2001, 20, 340-349.

[5] R. Stoll, A. Bosserhoff Curr. Protein. Pept. Sci. 2008 3, 221-226.

[6] J. E. Swedberg, C. I. Schroeder, J. M. Mitchell, T. Durek, D. P. Fairlie, D. J. Edmonds, D. A. Griffith, R. B. Ruggeri, D. R. Derksen, P. M. Loria, S. Liras, D. A. Price, D. J. Craik Eur. J. Med. Chem. 2015, 103, 175-184.

[7] J. W. Neidigh, R. M. Fesinmeyer, N. H. Andersen. Nat. Struct. Biol. 2002, 9, 425-430.

[8] R. W. Cheloha, A. Maeda, T. Dean, T. J. Gardella, S. H. Gellman. Nat. Biotechnol. 2014, 32, 653-655.

[9] G. L. Verdine, G. J. Hilinski Methods Enzymol. 2012, 503, 3-33.

[10] L. D. Walensky, G. H. Bird J. Med. Chem. 2014, 57, 6275-6288.

[11] A. Patgiri, M. Z. Menzenski, A. B. Mahon, P. S. Arora Nature Protocols 2010, 5, 1857-1865.

[12] H. E. Stanger, S. H. Gellman J. Am. Chem. Soc. 1998, 120, 4236-4237.

[13] A. G. Cochran, N. J. Skelton, M. A. Starovasnik Proc. Natl. Acad. Sci. U.S.A. 2001, 98, 5578-5583.

[14] B. L. Kier, J. M. Anderson, N.H. Andersen J. Am. Chem. Soc. 2015, 137, 5363-5371.

[15] P.-N. Cheng, C. Liu, M. Zhao, D. Eisenberg, J. S. Nowick Nature Chemistry 2012, 4, 927-933.

[16] R. N. Zuckermann, J. M. Kerr, S. B. H. Kent, W. H. Moos J. Am. Chem. Soc. 1992. 114, 10646-10647.

[17] A. S. Cu R. J. Ouellette Molecules 2010, 15, 5282-5335.

[18] T. W. Craven, M. Cho, N. J. Traaseth, R. Bonneau, K. Kirshenbaum J. Am. Chem. Soc. 2016, 138, 1543-1550.

[19] Thr and Glu have ΔΔG's of PPII-helix formation of 0.41 kcal; mol and −0.03 kcal/mol, respectively, based upon host-guest studies^{[20]}

[20] A. M. Brown, N. J. Zondlo Biochemistry 2012, 51, 5041-5051.

[21] G. L. Butterfoss, P. D. Renfrew, B. Kuhlman, K. Kirshenbaum, R. Bonneau J. Am. Chem. Soc. 2009, 131, 16798-16807.

[22] N-aryl peptoids preference PPII^{[22]}, (R/S)—N-(1-phenylethyl)^{[23]} and (R/S)—N-(1-naphthylethyl)^{[24]} preference PPI-helical conformations.

[23] N. H., Shah. G. L. Butter:foss, K. Nguyen, B. Yoo, R. Bonneau, D. L. Rabenstein, K. Kirshenbauml. J. Am. Chem. Soc. 2008, 130, 166222-16632.

[24] K. Kirshenbauin, A. E. Barron, R. A. Goldsmith, P. Armand, E. K. Bradley, K. T. V. Truong, K. A. Dill, F. E. Cohen, R. N. Zuckermann Proc. Natl. Acad. Sci. U.S.A. 1998. 95. 4303-4308.

[25] J. R. Stringer, J. A. Crapster, I. A. Guzei, and H. E. Blackwell J. Am. Chem. Soc. 2011, 133, 15559-15567.

[26] K. Drew, P. D. Renfrew, T. W. Craven, G. L. Butterfoss, F. Chou, S. Lyskov, B. N. Bullock, A. Watkins, J. W. Labonte, M. Pacella, K. P. Kilambi, A. Leaver-Fay, B. Kuhlman, J. J. Gray, P. Bradley, K. Kirshenbaum, P. S. Arora, R. Das, R. Bonneau PloS One 2013, e67051.

[27] P. D. Renfrew, T. W. Craven, G. L. Butterfoss, K. Kirshenbaum, R. Bonneau J. Am. Chem. Soc. 2014, 136, 8772-8782.

[28] J. T. Nguyen, C. W. Turck, F. E. Cohen, R. N. Zuckermann, W. A. Lim Science 1998. 282, 2088-2092.

[29] H. K. Ganguly, B. Majumder, S. Chattopadhyay, P. Chakrabarti, G. Basu J. Am. Chem. Soc. 2012, 134, 4661-4669.

[30] N. J. Zondlo Acc. Chem. Res. 2013, 46, 1039-1049.

[31] M. A. Kelly, B. W. Chellgren, A. L. :Rucker, J. M. Troutman, M. G. Fried, A. F. Miller, T. P. Creamer Biochemistry 2001, 40, 14376-14383.

[32] S. K. Holmgren, K. M. Taylor, L. E. Bretscher, R. T. Raines Nature 1998, 392, 666-667.

[33] P. Wilhelm, B. Lewandowski, N. Trapp, H. Wennemers J. Am. Chem. Soc. 2014, 136, 15829-15832.

[34] S. C. Lovell, J. M. Word, J. S. Richardson, D. C. Richardson Proteins: Struct. Funct. Bioinf. 2000, 40, 389-408.

[35] A. Leaver-Fay et al. An Object-Oriented Software Suite for the Simulation and Design of Macromolecules. In Methods in Enzymology; Johnson, M. L., Brand, L., Eds.; Computer Methods, Part C; Academic Press: New York, 2011; Vol. 487, pp 545-574.

[36] Z. E. Reinert, G. A. Lengyel, W. S. Horne, J. Am. Chem. Soc. 2013, 135, 12528-12531.

[37] J. D. Steinkruger, G. J. Bartlett, D. N. Woolfson, S. H. Gellman J. Am. Chem. Soc. 2012, 134, 15652-15655.

What is claimed is:

1. A miniature protein scaffold comprising Arg Val Arg Val Arg Thr Ser Arg Xaa Gly Tyr Asn Gly Thr Trp Ser Glu Trp Ser, wherein Xaa at position 9 is proline (Pro) or D-Pro and at least one of
the arginine (Arg) at position 1, 4, or 8;
the valine (Val) at position 2 or 4;
the threonine (Thr) at position 6 or 14;
the serine (Ser) at position 7, 16, or 19;
the tyrosine (Tyr) at position 11;
the asparagine (Asn) at position 12;
the glycine (Gly) at position 13; and
the glutamic acid (Glu) at position 17;
is replaced by an abiotic monomer, provided that the abiotic monomer is other than D-Pro or a substituted Pro residue.

2. A miniature protein scaffold comprising Arg Cys Arg Val Arg Thr Ser Arg D-Pro Gly Tyr Asn Gly Thr Trp Ser Glu Trp Cys, wherein at least one of
the arginine (Arg) at position 1, 4, or 8;
the valine (Val) at position 4;
the threonine (Thr) at position 6 or 14;
the serine (Ser) at position 7 or 16;
the tyrosine (Tyr) at position 11;
the asparagine (Asn) at position 12;
the glycine (Gly) at position 13; and
the glutamic acid (Glu) at position 17;
is replaced by an abiotic monomer, provided that the abiotic monomer is other than D-Pro or a substituted Pro residue.

3. The miniature protein scaffold of claim 2, wherein the cysteines (Cys) at positions 2 and 19 are linked via a disulfide bridge.

4. The miniature protein scaffold of any one of claims 1-3, wherein the abiotic monomer is selected from an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

5. The miniature protein scaffold of any one of claims 1-4, wherein the abiotic monomer is selected from an N-substituted glycine, a beta-peptoid, a beta-peptide, and a gamma-peptide.

6. The miniature protein scaffold of any one of claims 1-5, wherein the abiotic monomer is an N-substituted glycine.

7. The miniature protein scaffold of any one of claims 1-6, wherein at least one of the threonine (Thr) at position 14 and the glutamic acid(Glu) at position 17 is replaced by the abiotic monomer.

8. The miniature protein scaffold of any one of claims 1-7, wherein the Thr at position 14 is replaced by an N-substituted glycine or an N-substituted amino acid.

9. The miniature protein scaffold of any one of claims 1-7, wherein the Glu at position 17 is replaced by an N-substituted glycine or an N-substituted amino acid.

10. The miniature protein scaffold of any one of claims 1-7, wherein the Thr at position 14 and the Glu at position 17 are replaced by an N-substituted glycine or an N-substituted amino acid.

11. The miniature protein scaffold of any one of claims 1-10, wherein the abiotic monomer is an N-substituted glycine selected from the group consisting of N-(methoxyethyl) glycine, N—(S/R)-phenylethylamine glycine, N-(aryl) glycine, and N-(alkyl) glycine.

12. The miniature protein scaffold of any one of claims 1-11, wherein the abiotic monomer is N-methoxyethyl glycine.

13. The miniature protein scaffold of claim 2 comprising Arg Cys Arg Val Arg Thr Ser Arg D-Pro Gly Tyr Asn Gly Xaa Trp Ser Glu Trp Cys, wherein Xaa is N-methoxyethyl glycine.

14. The miniature protein scaffold of claim 2 comprising Arg Cys Arg Val Arg Thr Ser Arg D-Pro Gly Tyr Asn Gly Thr Trp Ser Xaa Trp Cys, wherein Xaa is N-methoxyethyl glycine.

15. The miniature protein scaffold of claim 2 comprising Arg Cys Arg Val Arg Thr Ser Arg D-Pro Gly Tyr Asn Gly Pro Trp Ser Xaa Trp Cys, wherein Xaa is N-methoxyethyl glycine.

16. The miniature protein scaffold of claim 2 comprising Arg Cys Arg Val Arg Thr Ser Arg D-Pro Gly Tyr Asn Gly Xaa Trp Ser Pro Trp Cys, wherein Xaa is N-methoxyethyl glycine.

17. The miniature protein scaffold of claim 2 comprising Arg Cys Arg Val Arg Thr Ser Arg D-Pro Gly Tyr Asn Gly Xaa Trp Ser Xaa Trp Cys, wherein Xaa is N-methoxyethyl glycine.

18. The miniature protein scaffold of claim 1 comprising Arg Val Arg Val Arg Thr Ser Arg D-Pro Gly Tyr Asn Gly Xaa Trp Ser Glu Trp Ser, wherein Xaa is N-methoxyethyl glycine.

19. The miniature protein scaffold of claim 1 comprising Arg Val Arg Val Arg Thr Ser Arg D-Pro Gly Tyr Asn Gly Thr Trp Ser Xaa Trp Ser, wherein Xaa is N-methoxyethyl glycine.

20. The miniature protein scaffold of claim 1 comprising Arg Val Arg Val Arg Thr Ser Arg D-Pro Gly Tyr Asn Gly Pro Trp Ser Xaa Trp Ser, wherein Xaa is N-methoxyethyl glycine.

21. The miniature protein scaffold of claim 1 comprising Arg Val Arg Val Arg Thr Ser Arg D-Pro Gly Tyr Asn Gly Xaa Trp Ser Pro Trp Ser, wherein Xaa is N-methoxyethyl glycine.

22. The miniature protein scaffold of claim 1 comprising Arg Val Arg Val Arg Thr Ser Arg D-Pro Gly Tyr Asn Gly Xaa Trp Ser Xaa Trp Ser, wherein Xaa is N-methoxyethyl glycine.

23. The miniature protein scaffold of claim 4, wherein the N-substituted amino acid selected from the group consisting of N-methyl alanine, N-methyl phenylalanine, N-methyl valine, N-methyl leucine, N-methyl isoleucine, N-methyl tryptophan, N-methyl methionine, N-methyl aspartic acid, N-methyl glutamic acid, N-methyl glycine, N-methyl serine, N-methyl threonine, N-methyl cysteine, N-methyl tyrosine, N-methyl asparagine, N-methyl glutamine, N-methyl lysine, N-methyl arginine, and N-methyl histidine.

24. The miniature protein scaffold of claim 4, wherein the side chain variant of Pro is selected from the group consisting of 4-fluoro-proline, 3-hydroxy-proline, 4-cyano-proline, 4-amino-proline, 3,4-dehydro-proline, azetidine-2-carboxylic acid, pipecolic acid, 4-oxa-proline, 3-thia-proline, and 4-[2-(trifluoromethyl)benzyll-proline.

25. A miniature protein scaffold comprising Arg Cys Arg Val Arg Thr Ser Arg Xaa Gly Tyr Asn Gly Thr Trp Ser Glu Trp Cys, wherein Xaa is proline (Pro) or D-Pro, the cysteines (Cys) at positions 2 and 19 are optionally linked via a disulfide bridge, and at least one of the threonine (Thr) at position 14 and the glutamic acid(Glu) at position 17 is replaced by an abiotic monomer, provided that the abiotic monomer is other than D-Pro or a substituted Pro residue.

26. The miniature protein scaffold of claim 25, wherein the Thr at position 14 is replaced by an abiotic monomer selected from the group consisting of an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

27. The miniature protein scaffold of claim 25, wherein the Glu at position 17 is replaced by an abiotic monomer selected from the group consisting of an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

28. The miniature protein scaffold of claim 25, wherein the Thr at position 14 and the Glu at position 17 are replaced by an abiotic monomer selected from the group consisting of an N-substituted glycine, an N-substituted amino acid, a side chain variant of Pro, a beta-peptoid, a beta-peptide, a gamma-peptide, a pyridine oligoamide, a quinoline amide-linked monomer, an aryl amide-linked monomer, an aedemer, a peptide nucleic acid, a monomer constituting oligoamide folded oligomers, a sulfonamidopeptide, an aminoxy acid monomer, a hydrazone-linked pyrimidine, an ureidophthalimide monomer, a triazine-based monomer, a triazole-based monomer, an oxopiperazine monomer, and a urea-linked monomer.

29. The miniature protein scaffold of any one of claims 25-28, wherein the abiotic monomer is selected from an N-substituted glycine, a beta-peptoid, a beta-peptide, and a gamma-peptide.

30. The miniature protein scaffold of any one of claims 25-29, wherein the abiotic monomer is an N-substituted glycine.

31. The miniature protein scaffold of any one of claims 26-30, wherein the N-substituted glycine is selected from the group consisting of N-(methoxyethyl) glycine, N—(S/R)-phenylethylamine glycine, N-(aryl) glycine, and N-(alkyl) glycine.

32. The miniature protein scaffold of claim 31, wherein the N-substituted glycine is N-methoxyethyl glycine.

33. The miniature protein scaffold of any one of claims 26-28, wherein the N-substituted amino acid is selected from the group consisting of N-methyl alanine, N-methyl phenylalanine, N-methyl valine, N-methyl leucine, N-methyl isoleucine, N-methyl tryptophan, N-methyl methionine, N-methyl aspartic acid, N-methyl glutamic acid, N-methyl glycine, N-methyl serine, N-methyl threonine, N-methyl cysteine, N-methyl tyrosine, N-methyl asparagine, N-methyl glutamine, N-methyl lysine, N-methyl arginine, and N-methyl histidine.

34. The miniature protein scaffold of any one of claims 26-28, wherein the side chain variant of Pro is selected from the group consisting of 4-fluoro-proline, 3-hydroxy-proline, 4-cyano-proline, 4-amino-proline, 3,4-dehydro-proline, azetidine-2-carboxylic acid, pipecolic acid, 4-oxa-proline, 3-thia-proline, and 4[2-(trifluoromethy)benzyl]-proline.
